(19) United States
(12) Patent Application Publication
(10) Pub. No.: US 2022/0090166 A1
(43) Pub. Date: Mar. 24, 2022

(54) PREPARATION METHODS AND APPARATUS ADAPTED TO FILTER SMALL NUCLEIC ACIDS FROM BIOLOGICAL SAMPLES

(71) Applicant: Siemens Healthcare GmbH, Erlangen (DE)

(72) Inventors: Yiwei Huang, Erlangen (DE); Guido Hennig, Köln (DE)

(73) Assignee: Siemens Healthcare GmbH, Erlangen (DE)

(21) Appl. No.: 17/310,474

(22) PCT Filed: Mar. 3, 2020
(86) PCT No.: PCT/US20/20723

§ 371 Date: Aug. 5, 2021

Related U.S. Application Data
(60) Provisional application No. 62/813,391, filed on Mar. 4, 2019.

(51) Int. Cl. C12Q 1/6806; C12N 15/10

(52) U.S. Cl. CPC C12Q 1/6806; C12N 15/1013

(57) ABSTRACT

Sample preparation methods enabling selective and enriched extraction of small nucleic acid fragments from biological samples. The methods include adding lysed sample, first magnetic particles, and first binding buffer in a first vessel and incubating to bind first nucleic acid portion of lengths ≥500 bp to the first magnetic particles and leave a first supernatant. First supernatant is transferred to a second vessel with second magnetic particles and a second binding buffer and then incubated to bind a second nucleic acid portion having lengths <500 bp to the second magnetic particles and leave a second supernatant. Second magnetic particles with bound second nucleic acid portion are separated and washed. An elution buffer is added to the second magnetic particles and incubated to release the second nucleic acid portion (<500 bp) and form a final eluate. Final eluate can be processed such as by using RT-PCR and PCR.

### RELATED APPLICATIONS

This application claims priority to U.S. Provisional Application Ser. No. 62/813,391 entitled “METHOD FOR FILTERING SMALL NUCLEIC ACIDS, IN PARTICULAR FROM SERUM OR PLASMA SAMPLES” filed on Mar. 4, 2019, the disclosure of which is hereby incorporated by reference in its entirety herein.

### FIELD

The present disclosure relates to sample preparation methods, and kits used to extract nucleic acids, such as in preparation for molecular assays (e.g., via polymerase chain reaction (PCR) or RT-PCR testing).

### BACKGROUND

When cells become apoptotic, their nucleic acids are fragmented to a specific size and released into the bloodstream. Some such nucleic acid fragments are referred to as cell-free DNAs (hereinafter “cfDNA”). RNA fragments are also present. The cfDNA and RNA fragments remain as circulating fragments in the blood for some time. Like other blood analytes, such nucleic acids can be readily accessed by way of blood sampling by a phlebotomist.

A wide variety of diagnostic instruments (e.g., molecular analysis instruments) are used to analyze patient specimens (biological samples and nucleic acids such as DNA and RNA therein). These diagnostic instruments may conduct an assay (e.g., a molecular assay) using magnetic particles as a binding support, lysis and elution buffers, or other additives to identify a constituent (e.g., nucleic acid) in, or characteristic of, a patient sample. Some molecular assay apparatus may use PCR, wherein a sample preparation method providing nucleic acid extraction is used. Once the nucleic acid is extracted by the sample preparation method, an amplification and detection device of the PCR apparatus may be used to replicate (amplify) and measure the extracted DNA and/or RNA templates from processed eluate derived from the biological samples by the sample preparation method. In some cases, it may be desirable to preferentially extract certain nucleic acids (e.g., cfDNA) from the sample and further replicate and analyze these cfDNA fragments. However, it has been a significant challenge in the art to extract such cfDNA fragments.

Therefore, preparation methods, kits, and sample preparation apparatus that improve efficiency of extraction and/or amount of cfDNA extracted in sample preparation (e.g., in preparation for PCR processing) are desired.

### SUMMARY

According to a first aspect, a method of extracting nucleic acids from a biological sample is provided. The sample preparation method includes providing a sample portion of the biological sample containing the nucleic acids to a first vessel; causing lysis of the sample portion to form a lysed sample; adding first magnetic particles to the lysed sample along with a first binding buffer to form a first bindable mixture; incubating the first bindable mixture in a first incubation to bind a first nucleic acid portion having lengths greater than or equal to 500 bp to the first magnetic particles and leave a first supernatant; separating the first magnetic particles from the first supernatant; adding second magnetic particles to the first supernatant along with a second binding buffer to form a second bindable mixture; incubating the second bindable mixture in a second incubation to bind a second nucleic acid portion having lengths less than 500 bp to the second magnetic particles and leave a second supernatant; separating the second magnetic particles with the second nucleic acid portion bound thereto from the second supernatant; washing the second magnetic particles with second nucleic acid portion bound thereto; and adding an elution buffer to the second magnetic particles and incubating in a third incubation to release the second nucleic acid portion and from a third supernatant.

In another aspect, a kit adapted to preparation of a biological sample for further molecular diagnostic processing (e.g., for further PCR processing) is provided. The kit includes a lysis buffer configured to lyse the biological sample; a first binding buffer comprising one or more chaotropic agents, a salt compound, and a surfactant; a second binding buffer comprising: an alcohol comprising isopropanol, ethanol, or a combination thereof, and a salt compound comprising sodium chloride, potassium chloride, sodium phosphate, potassium phosphate, or a combination thereof; magnetic particles operable as binding supports; a first wash buffer comprising a chaotropic agent, a salt compound, and an alcohol; a second wash buffer comprising a salt compound and an alcohol; and an elution buffer comprising TRIS-HCL.

According to yet another aspect, a sample preparation system adapted to prepare a biological sample for molecular processing is provided. The sample preparation system includes a kit comprising a lysis agent, a first binding buffer comprising one or more chaotropic agents, a salt compound, and a surfactant, a second binding buffer comprising: an alcohol comprising isopropanol, ethanol, or a combination thereof, and a salt compound comprising sodium chloride, potassium chloride, sodium phosphate, potassium phosphate, or a combination thereof, magnetic particles operable as binding supports, a first wash buffer comprising a chaotropic agent, a salt compound, and an alcohol, a second wash buffer comprising a salt compound and an alcohol, and an elution buffer comprising TRIS-HCL; a first vessel positioned to receive a sample portion of the biological sample containing nucleic acids and the lysis agent; a heater element operable to heat the sample portion and lysis agent and form a lysed sample; a pipette coupled to an aspiration and dispensing apparatus and configured and operable to aspirate and dispense the first magnetic particles and the first binding buffer into the lysed sample and form a first bindable mixture, which upon a first incubation binds a first nucleic acid portion having lengths greater than or equal to 500 bp to the first magnetic particles and leaves a first supernatant; a first magnet operable to separate the first magnetic particles with bound first nucleic acid portion from the first supernatant; a second vessel receiving the first supernatant, the second magnetic particles, and the second binding buffer, which upon a second incubation binds a second nucleic acid portion having lengths less than 500 bp to the second magnetic particles and leaves a second supernatant; a second magnet separating the second magnetic particles with the second nucleic acid portion bound thereto from the second supernatant; a wash station configured to carry out first and second wash phases of the second magnetic particles with bound second nucleic acid portion, after separation from the second supernatant, wherein the first wash phase comprises immersing the second magnetic particles with a first wash buffer and the second wash phase comprises immersing the second magnetic particles with a second wash buffer; and an elution stage wherein the elution buffer is added to the second magnetic particles after the first and second wash phases and incubated in a third incubation to release the second nucleic acid portion and form final eluate.

Still other aspects, features, and advantages of the present disclosure may be readily apparent from the following detailed description by illustrating a number of example embodiments, including the best mode contemplated for carrying out the present invention. The present disclosure may also be capable of other embodiments, and its several details may be modified in various respects, all without departing from the scope of the present disclosure. Accordingly, the drawings and descriptions are to be regarded as illustrative in nature, and not as restrictive. The disclosure is to cover all modifications, equivalents, and alternatives falling within the scope of the claims.

### BRIEF DESCRIPTION OF THE DRAWINGS

The drawings, described below, are for illustrative purposes only and are not necessarily drawn to scale. The drawings are not intended to limit the scope of this disclosure in any way.

FIGS. 1A-1M illustrate schematic side views of the various sequences of the sample preparation method enabling preferential extraction of small nucleic acids according to one or more embodiments of the disclosure.

FIGS. 1N-1P illustrates schematic side views of various sequences of a further molecular processing and analysis (e.g., PCR processing and testing) enabling replication and testing of such small nucleic acids (e.g., <500 bp) according to one or more embodiments of the disclosure.

FIG. 2 illustrates a schematic diagram of a sample preparation system configured to extract small nucleic acids according to one or more embodiments of the disclosure.

FIG. 3 illustrates a flowchart of a method of extracting small nucleic acids from a biological sample according to one or more embodiments of the disclosure.

FIG. 4 illustrates a flowchart of a PCR method according to one or more embodiments of the disclosure.

### DETAILED DESCRIPTION

Reference will now be made in detail to the various embodiments of this disclosure, examples of which are illustrated in the accompanying drawings. Wherever possible, the same reference numbers will be used throughout the drawings to refer to the same or like parts.

When cells become apoptotic, a form of programmed cell death, their nucleic acids (e.g., DNA) are fragmented to specific-size nucleic acid fragments of from about 160 bp to about 180 bp in base length, and released into the bloodstream. In particular, apoptosis is an orderly process in which the cell's contents break down and are packaged into small packets of membrane (e.g., referred to herein as cell-free DNAs or cfDNA) for ultimate collection by the immune cells. These cfDNA packets (as well as RNA) remain as circulating fragments in the blood for some time and, like other blood analytes, can be assessed by blood sampling. The cfDNA may have a half-life of about two hours in blood, for example. Thus, as long as they can be processed quickly, the cfDNA fragments can be used for blood analysis.

Today, cancer is one of the leading causes of death worldwide, and thus improved diagnostic methods are needed to accurately and rapidly provide a diagnosis thereof. In some cases, solid biopsies of affected tissue can be conducted. However, such solid biopsies may not be preferred because they: 1) have an invasive character, and 2) cannot, or only poorly, reflect current tumor dynamics or sensitivity to treatment.

On the other hand, a liquid biopsy can be readily obtained. Further, it is generally understood that the amount of cfDNA correlates to the total amount of tumor distributed throughout the body. Thus, it can be therefore a measure of tumor burden, and provide for analysis of specific cancer mutations. While cfDNA is detected in the blood of normal subjects at levels that range from 36 ng/mL to 156 ng/mL, it is found to be elevated in the blood of cancer patients to levels that can range from 58 ng/mL to 5317 ng/mL. See Schwarzenbach H, Pantel K, Kemper B, Beeger C, Otterbach F, Kimmig R, Kasimir-Bauer S, “Comparative evaluation of cell-free tumor DNA in blood and disseminated tumor cells in bone marrow of patients with primary breast cancer”; Breast Cancer Res. 2009; 11(5):R71; and Madhavan D, Wallwiener M, Bents K, Zucknick M, Nees J, Schott S, Cuk K, Riethdorf S, Trumpp A, Pantel K, Sohn C, Schneeweiss A, Surowy H, Burwinkel B, “Plasma DNA integrity as a biomarker for primary and metastatic breast cancer and potential marker for early diagnosis”; Breast Cancer Res Treat. 2014 July; 146(1):163-74.

Thus, testing based on identification of cfDNA in the obtained biological sample has the potential for early detection of specific genetic and/or epigenetic mutations. Thus, cfDNA analysis has the possibility of providing improved cancer screening. Moreover, cfDNA analysis may offer the possibility of providing improved cancer therapy that is guided by the identification of certain actionable mutations. Since most cfDNA stems from healthy human cells, tumor cfDNA is usually only available in traces. Obtaining these traces of such tumor-specific nucleic acids is still a substantial challenge. Detection of tumor mutations is a challenge even in the <500 bp fraction, but presence of high molecular weight fragments ≥500 bp) in the final eluate can cause so much background noise that it can partially or even fully obscure the signal from the <500 bp fraction containing the mutations. Thus, the inventors determined it is desirable to remove as much of the ≥500 bp fraction as possible.

Thus, in accordance with a first aspect, the present disclosure provides an improved method of filtering (extracting) these small nucleic acid fragments (e.g., cfDNA) from a portion of a biological sample (e.g., blood), such as from serum or plasma thereof. RNA may also be extracted using the method. The method disclosed herein may enable extraction of relatively more and/or relatively more pure cfDNA. In particular, in one or more embodiments, a two-part purification method is provided, which employs magnetic particles (e.g., silica coated magnetic beads) in a first binding step to first extract (or filter) high molecular weight fraction nucleic acids. High molecular weight nucleic acids (e.g., cfDNA) having lengths of ≥500 bp originate mostly from healthy white blood cells (e.g. centrifugation leakage) and do not contain any significant mutations. Removing a substantial portion of the high molecular weight fraction of nucleic acids can reduce costs and was discovered that it increase analytical sensitivity of detection of genetic and/or epigenetic mutations.

According to the first part of the disclosed method, the high molecular weight nucleic acid fragments, i.e., those fragments with large numbers of base pairs (e.g., fragments with lengths greater than or equal to 500 bp) will bind to first magnetic particles and will be removed from the portion of the biological sample in the first part. In the second part of the two-part method, the small nucleic acid fragments (e.g., of less than 500 bp in base length), which were retained after completing the first step, will bind to second magnetic particles and will be purified from the sample-portion containing solution. Utilizing the two-part method, the resulting extracted nucleic acids (e.g., cfDNA) can be much purer, i.e., has less remaining large nucleic acid (≥500 bp) contamination than previous methods. Moreover, because the present method enables extraction of relatively more pure nucleic acids (e.g., cfDNA) it can thus can provide improved signal detection thereof.

Once the small nucleic acids templates (<500 bp) are extracted, they may be replicated (amplified) using any suitable molecular assay technology such as PCR, or RT-PCR, isothermal DNA amplification, multiple displacement amplification, and/or other known replication methods. Accordingly, one or more embodiments of the disclosure provide sample preparation methods, kits, and sample preparation sysems adapted to enable the ability to yield higher levels of cfDNA and/or much purer cfDNA, while having relatively low levels of high molecular weight DNA (e.g., DNA fragments having lengths ≥500 bp).

Thus, in a first broad aspect, sample preparation methods are provided. After cell lysis, a two-step sample preparation method is used to isolate certain small nucleic acids (e.g., cfDNA). The two-step method involves a first negative-selection binding step wherein relatively-high molecular weight nucleic acids (≥500 bp) are partly removed (e.g., 50% or more). The high molecular weight DNA (≥500 bp) is waste to be removed because the inventors have recognized that it tends to generate extremely-high background noise as compared to the amount of targeted nucleic acids (e.g., cfDNA including target/mutated cancers) that are present. Further the presence of high molecular weight nucleic acids (≥500 bp DNA and RNA) can generate relatively high sequencing costs in next generation sequencing experiments/analyses.

Therefore, in accordance with embodiments, sample preparation methods, kits, apparatus, and systems that can be used to effectively isolate high-quality nucleic acids (e.g., cfDNA) are provided. In a first aspect, a method of extracting small nucleic acids from a biological sample is provided. The method involves providing a portion of the biological sample in a first vessel and lysis of that sample portion to form a lysed sample. First magnetic particles are added to the lysed sample along with a first binding buffer to form a first bindable mixture. This first bindable mixture is incubated in a first incubation to bind relatively large nucleic acids (≥500bp DNA and RNA) to the first magnetic particles and leave behind a first supernatant. The first magnetic particles are separated from the first supernatant, and second magnetic particles are then added to the first supernatant along with a second binding buffer to form a second bindable mixture. Second bindable mixture is incubated in a second incubation to bind small nucleic acids (e.g., cfDNA) having lengths less than 500 bp to the second magnetic particles and leave a second supernatant. The second magnetic particles with bound small nucleic acids are then separated from the second supernatant. Following washing (e.g., a two-phase wash) of the second magnetic particles with bound small nucleic acids, an elution buffer is added to the second magnetic particles and incubation in a third incubation is undertaken to release the small nucleic acids having lengths less than 500 bp from the second magnetic particles and form a third supernatant (final eluate). The third supernatant may then be further processed (e.g., amplified) by known molecular processing (e.g., PCR processing or the like) and then the amplified small nucleic acid templates having lengths less than 500 bp may be analyzed for size, quantity, and/or sequence. Further, fluorescent spectroscopy utilizing fluorescently-labeled probes or fluorescently-labeled primers may be used to facilitate the analysis.

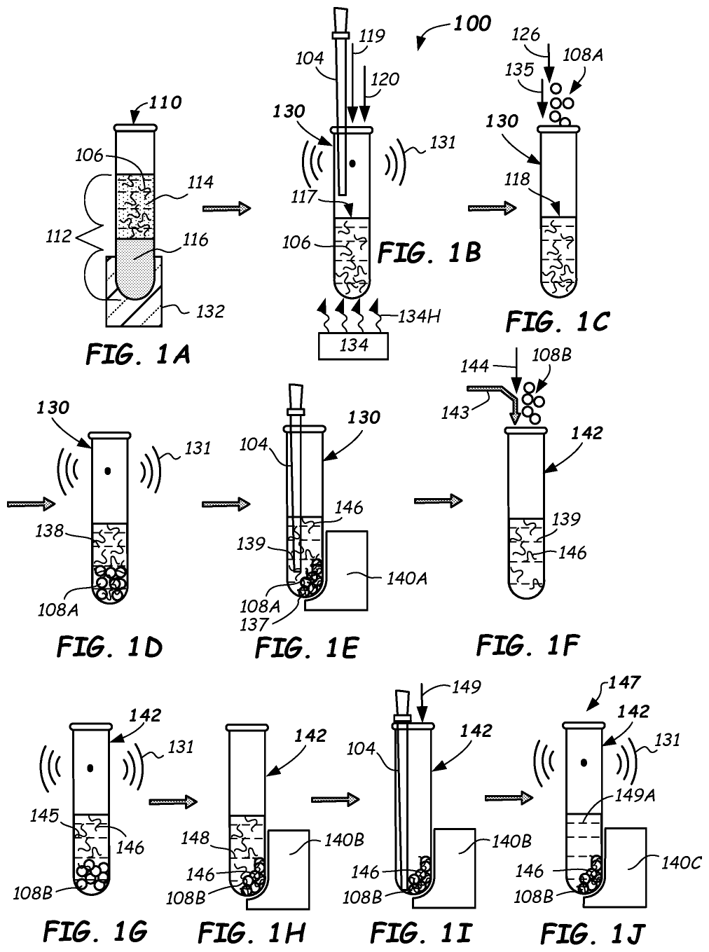

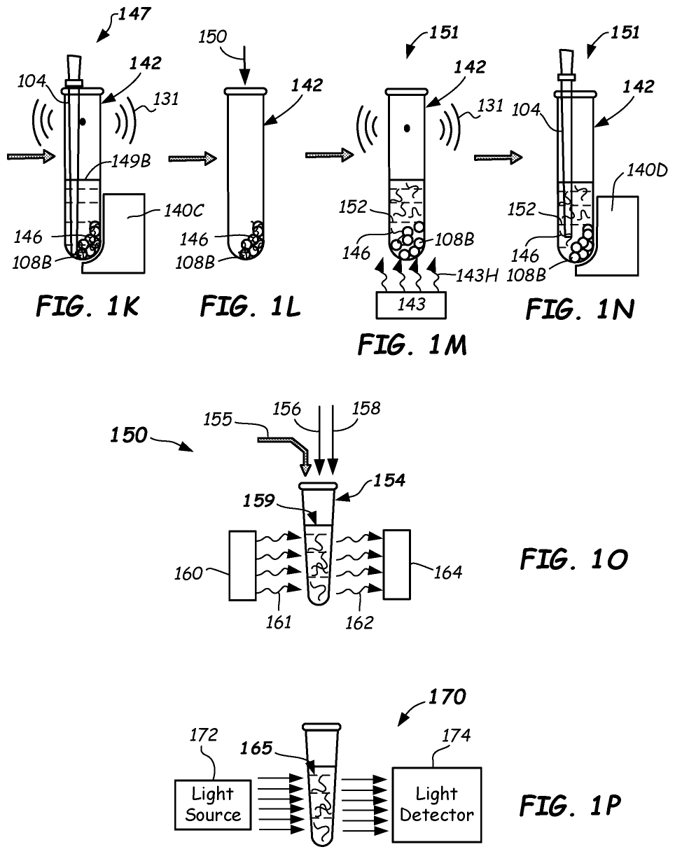

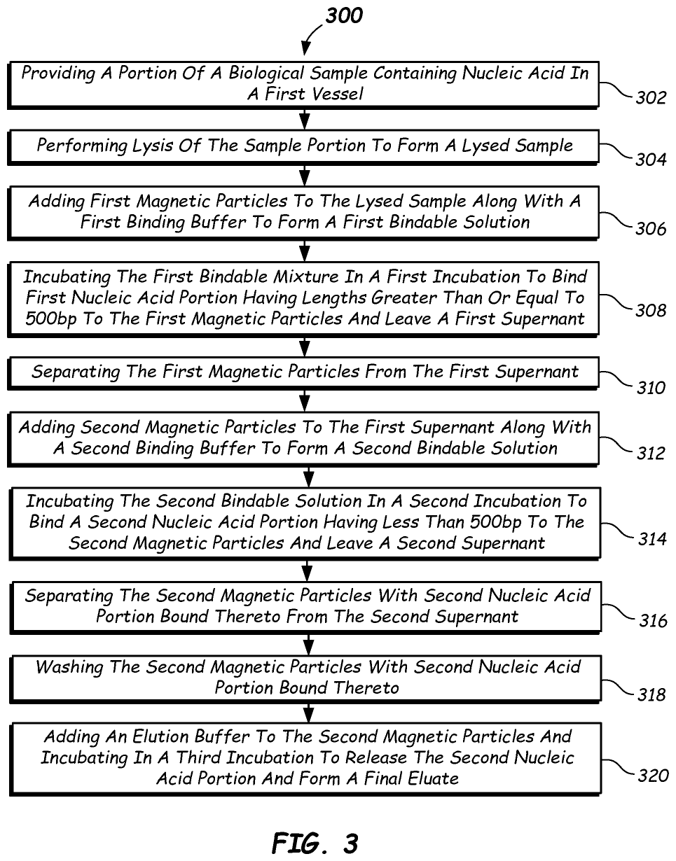

These and other aspects and features of embodiments of the disclosure are now described in full detail with reference to FIGS. 1A-4 herein.

FIGS. 1A-1N and FIGS. 2 and 3 will be referred to herein to fully explain sample preparation methods 300 that are adapted to preferentially extract small nucleic acids (e.g., cfDNA) having lengths less than or equal to 500 bp from a biological sample as well as a sample preparation system 200 adapted to automatically carry out the method 300 according to embodiments of the present disclosure. Optionally, the sample preparation method 300 may be carried out manually, as also disclosed herein.

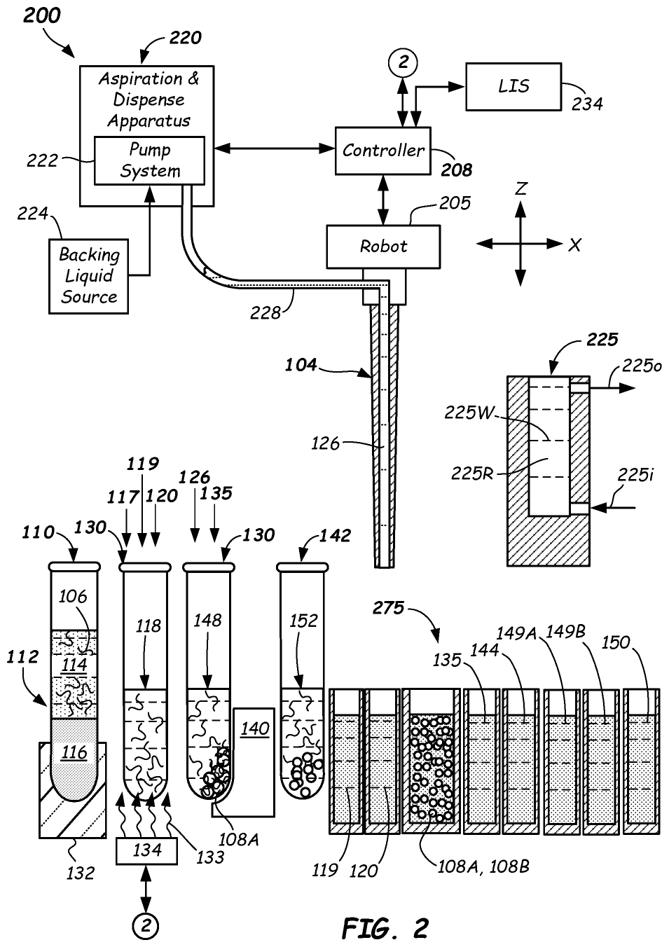

In a first aspect, as best shown in FIG. 2, the sample preparation system 200 that can be used for automatically carrying out the sample preparation method 300 includes various locations within the reach of a pipette 104, wherein the pipette 104 is moveable by a robot 205 coupled to the pipette 104 and wherein movement may be responsive to control signals provided by a controller 208. The controller 208 may include a suitable processor and memory. Processor may include any suitable microprocessor or other processing device adapted to execute software program instructions and interface with memory and various other components of the sample preparation system 200. For example, processor may be included in a windows-based computer, for example. Memory may be operative to store software code for carrying out the sample preparation method 300 as described herein, including code configured to operate of the robot 205 and other associated parts of the sample preparation system 200 (e.g., aspiration and dispense apparatus 220, heating elements 134, 143, magnets 140A-140D, agitation members, etc.). Controller 208 may also control various functions of an associated molecular processing and analysis apparatus (e.g., PCR apparatus), including an amplification apparatus 150 (FIG. 1O) that is adapted to carry out amplification of the nucleic acid templates by imparting multiple heating and cooling cycles, and the analysis apparatus 170 (FIG. 1P) that is adapted to measure emissions (e.g., fluorescent emissions) from a PCR solution, and other conventional molecular analysis apparatus (e.g., PCR apparatus and the like).

According to the sample preparation method 300, a biological sample 112 can be provided in a sample collection tube 110. For example, sample collection tube 110 may be a vacuum blood collection tube with drawn whole blood therein. The sample collection tube 110 may also include an anti-coagulant such as ethylenediamine tetraacetic add (EDTA) therein, in some embodiments. Other suitable anti-coagulants for hematological testing may be used that allow preservation of cellular components and morphology of blood cells. Biological sample 112 may be a fractionated (centrifuged) biological sample. In the case of whole blood, the biological sample 112 can be made up of a serum or plasma portion 114 and a settled red blood cell portion 116 after fractionation. Centrifugation of the biological sample 112 can be for about 10 minutes at 2000×G, for example, to bring about the fractionation. Other suitable centrifugation processes can be used.

The serum or plasma portion 114, after fractionation, contains nucleic acids 106 including the small nucleic acids (e.g., cfDNA) that are to be preferentially extracted according to the sample preparation method 300. The present embodiment of the sample preparation method 300 will be described with reference to plasma comprising the serum or plasma portion 114. However, the present disclosure is equally applicable to serum comprising the serum or plasma portion 114. Additionally, the present sample preparation method 300 and sample preparation system 200 is also applicable to extracting small nucleic acids (e.g., cfDNA or RNA) from other suitable types of biological samples, such as from urine, saliva, cerebrospinal fluid, pleural fluid, or other biological fluids.

As should be understood, the sample preparation method 300 described herein can be performed manually or automatically or with any combination of the foregoing. Example automated and manual methods will be described herein. It should be understood that any automated method step described herein could optionally be performed manually.

In a first example sample preparation method 300, a first vessel 130 can be provided at a location accessible by the pipette 104, and a defined volume of a sample portion 117 of the serum or plasma portion 114 of the biological sample 112 containing nucleic acid (e.g., DNA and RNA) fragments 106 may be dispensed into the first vessel 130 by pipette 104. Optionally, the serum or plasma portion 114 may be transferred to an intermediate vessel, further centrifuged, and then the sample portion 117 can be transferred to the first vessel 130 either manually or in an automated manner via pipette 104 or other pipette. The defined volume of the sample portion 117 of the serum or plasma portion 114 of the sample 112 can be 3 mL of serum or plasma, for example, or other precisely-measured small volume (e.g., 10 mL). However, the method and kit can also be used for larger volume sample portions 117 of greater than 10 mL.

The dispensing can be automated such as by an aspiration from the sample collection tube 110 or other intermediate vessel (if used) and then the sample portion 117 can be then dispensed into the first vessel 130 by pipette 104 coupled to an aspiration and dispense apparatus 220. Aspiration and dispense apparatus 220 may include a pump system 222 coupled to a backing liquid source 224, wherein the backing liquid may be nuclease-free deionized water 126, for example. The nuclease-free deionized water 126 can be used as part of the method 300, as will be described herein. The pump system 222, which can include a precision pump, can be coupled to the pipette 104 by a flexible conduit 228 also containing the nuclease-free deionized water 126 as the backing liquid, for example. Any suitable aspiration and dispense apparatus 220 may be used for the aspiration and dispensing of sample portion 117, nuclease-free deionized water, and various liquid consumables (e.g., serine protease, lysis buffer, first and second binding buffers, magnetic particle suspensions, wash solutions, elution buffer, PCR master mix, primer or probe, and the like). More than one pipette can be used. For example, there may be a dedicated pipette for the sample portion 117, and one or more other pipettes for the other consumables. Suitable aspiration and dispense apparatus 220 are described, for example, in U.S. Pat. Nos. 5,777,221; 6,060,320; 6,158,269; 6,250,130; 6,463,969: 7,998,751; 7,205,158. Other suitable aspiration and dispensing apparatus may be used.

In some embodiments, the pipette 104 or other pipette may include a disposable pipette tip (not shown). Pipette tips may be replaced after each dispense from a supply of pipette tips that are accessible by the robot 205. Optionally, or additionally, the sample preparation system 200 may include one or more wash stations 225, each including a reservoir 225R configured to receive a wash liquid 225W therein. The one or more wash stations 225 are accessible by the pipette 104 and thus can wash the pipette 104 after each aspiration and dispense of a sample portion 117 and/or consumable liquid. Reservoir 225R can include a flow of wash solution 225W therein via inlet 225i coupled to a source of wash liquid (not shown) and outlet 225o.

First vessel 130 can be any suitable vessel, such as a centrifugation tube, cuvette, or a well of an extraction well plate. The first vessel 130 can have a volume capacity of about 15 mL or greater, for example. Other vessels sizes may be used. Thus, it should be understood that in some embodiments, the sample processing method 300 described herein can be performed in tandem within multiple wells of an extraction well plate. If the first vessel 130 comprises a well of an extraction well plate, then the extraction well plate may be a 96 well (e.g., 8×12), deep-well plate, for example.

In the case of preparation on an extraction well plate, following the carrying out of the sample processing method 300 according to the disclosure herein, the final eluted solution 152 (eluate) including the extracted small nucleic acids (e.g., cfDNA) that have lengths of less than 500 bp may be transferred to a test plate (not shown), which may be a PCR test plate (e.g., a 96 well test plate) for further PCR processing. The further processing may be to replicate (amplify) the extracted small nucleic acid templates that have length less than 500 bp and subsequently measure the progress of the PCR replication and/or measure fluorescent emissions at one or more wavelengths, or other analyses thereof. However, it should be apparent that the extraction well plate and the PCR test plate may have other configurations (e.g., different numbers of wells, or different numbers of rows and columns). Any suitable article including the first vessel 130 or configuration of the first vessel 130 may be used. In some embodiments, the further PCR processing after the completion of the sample preparation method 300, may involve transfer of the final eluate 152 for further molecular processing (e.g., PCR processing) on a single vessel.

In some embodiments, the sample preparation system 200 may further include one or more sample holders 132, such as one or more sample racks, that may be configured to hold sample collection tubes 110 that contain patient samples 112 wherein the patient samples 112 may have been obtained from multiple patients. In some embodiments, the sample holder(s) 132 containing a plurality of patient samples 112 from different patients may be loaded onto one or more autoload trays, and may be automatically loaded via a prompt or other action into the sample preparation system 200 of a molecular analysis apparatus (e.g., PCR instrument) at a location that is accessible by the pipette 104. Upon being loaded into the sample preparation system 200, a reader device may read a sample holder identifier and/or sample identifiers on each sample collection tubes 110. Thus, sample identification data on patient samples 112 and their location in the sample holder(s) 132 may be stored in memory of a controller 208 of the sample preparation system 200.

The controller 208 may also interface with a laboratory information system (LIS) 234 or another server or computer so that results of the molecular analysis apparatus (e.g., PCR instrument) can be conveyed to interested parties. LIS 234 may include a LIS communicator, a digital communication device that can interface and communicate digitally with controller 208. Controller 208 may receive input from the LIS 234 on what assays to run on each biological sample 112. Controller 208 may receive assay order information from the LIS communicator for various patient samples 112, and also return result files and/or other information to the LIS communicator and thus to the LIS 234. Communication between the LIS communicator and the controller 208 and LIS 234 may be by using any suitable communication protocol.

Following the provision of the sample portion 117 of the biological sample 112 containing nucleic acid 106 (E.g., DNA and RNA) into first vessel 130 in block 302, the method 300 further includes, in block 304, causing lysis of the sample portion 117 of the biological sample 112 as shown in FIG. 1B to form a lysed sample 118 (lysate) as shown in FIG. 1C. Lysis is a step in the sample preparation method 300 wherein proteins are isolated from their source. Lysis breaks down the cell membrane to separate the proteins and nucleic acids 106 from the non-soluble parts of the cell. Lysis is conducted by introducing a lysis buffer 119 to the sample portion 117 followed by a first incubation. Lysis buffer 119 can be added by the pipette 104 or a separate pipette (not shown). Lysis buffer 119 can optionally be added manually.

The lysis can be accomplished in chaotropic, high salt conditions to release nucleic acids 106 from the sample portion 117, as well as protect the nucleic acids 106 from cellular nucleases. Prior to isolation, the sample portion 117 of the serum or plasma portion 114 can be treated with a protein removal agent 120, such as serine protease. One such serine protease can be proteinase K, which is adapted to remove nucleic acid binding proteins. For example, the protein removal agent 120 (e.g., proteinase K) can be added to the dispensed sample portion 117 (serum or plasma portion) in a volume of from about 90 μL to about 110 μL, for example, or in an amount of from 30 μL to about 37 μL per 1 mL of the sample portion 117. The protein removal agent 120 can be added via an aspiration and dispense by pipette 104 or by another pipette. Protein removal agent 120 can be a consumable and can be stored locally at a position accessible by the pipette 104 or other pipette. For example, the protein removal agent 120 may be located at an access area that is configured to contain other consumables.

The consumables may include components that are used in various parts of the sample preparation method 300 or later on the replication (amplification) phase of molecular processing(e.g., PCR processing). Consumables may include, but are not limited to, vessels 130 (e.g., centrifugation tubes, cuvettes, multi-well plates, or the like), pipette tips, protein removal agent 120, lysis buffer 119, suspensions of magnetic beads 108A, 108B, first binding buffer 135, second binding buffer 144, wash buffers 149A, 149B, elution buffer 150, various calibrators, controls (e.g., pre-processed controls, post-processed controls, internal controls), primer or probe, master mixes, and/or other consumable processing components.

In the case of implementing the sample preparation method 300 on a multi-well extraction plate, and depending upon the number of different assays and/or assay types to be run, the extraction plate wells comprising the first vessels 130 and second vessels 142 may include multiple sample portions 117 that have been obtained from the same or different patients as well as control and/or calibrator samples.

The lysis buffer 119 can be any suitable compound that causes cell lysing and that may also stabilize proteins and prevent activity of RNase enzymes and DNase enzymes by denaturing them. In some embodiments, the lysis buffer 119 can include, for example, one or more chaotropic agents. The one or more chaotropic agents can comprise urea (CH₄N₂O), a guanidinium-based compound such as guanidine hydrochloride or guanidinium thiocyanate, or a combination of any of the foregoing. The concentration of the chaotropic agents can be from 2M to 6M, or even from 4M to 6M, in some embodiments.

In some embodiments, the lysis buffer 119 may comprise one or more chaotropic agents combined with a salt compound. The salt compound can function as a buffering agent during lysis to reach a desired ionic strength. A desired pH of the lysis buffer 119 can be from 4 to 7. The salt compound can comprise glycine hydrochloride, potassium hydrogen phthalate/hydrochloric acid (KHP-HCL), sodium citrate, sodium acetate, potassium hydrogen phthalate/sodium hydroxide (KHP-NaOH), sodium phosphate, potassium phosphate, Tris-HCL, and the like. The salt compound can be used in a concentration of from 50 mM and 150 mM, for example.

Lysis buffer 119 may additionally comprise a surfactant. A suitable surfactant can comprise a polyethylene glycol derivative (e.g., C₁₆H₂₆O₂) or polyoxyethylene sorbitol esteris, for example. The surfactant can be added in an amount from 5 vol. % to 15 vol. % and may be ionic, nonionic, or zwitterionic and can act as a detergent, dispersant to prevent aggregation, or an emulsifier.

Lysis buffer 119 can be added to the sample portion 117 and protein removal agent 120 in an amount of about 3.37 mL to about 4.13 mL, or in the amount of from 1.12 mL to about 1.38 mL per 1 mL of sample portion 117, for example. Lysis can be carried out by capping or covering and suitably mixing of the solution of sample portion 117, protein removal agent 120, and lysis buffer 119. Mixing (denoted by vibration 131) may take place in stages as individual components are added. Thereafter, the solution may be heated, such as in a thermostat or the like, by exposure to heat 134H from a heating element 134 for an effective amount of time as shown in FIG. 1B and FIG. 2. For example, the heating of the solution can be carried out in a lysis incubation at a temperature of from about 25° C. to about 45° C., for example. Any suitable heating method and apparatus may be used, such as by a dry block heater with thermostat. Lysis incubation of the solution may be carried out for a lysis period of between 8 minutes and 12 minutes, for example or until substantially complete lysis occurs.

Once the lysing step in block 304 is completed, in block 306 and FIG. 1C, first magnetic particles 108A are added to the lysed sample 118 along with nuclease-free deionized water 126 and a first binding buffer 135 to form a first bindable mixture 138 (see FIG. 1D). First magnetic particles 108A can have a mean particle diameter of from about 150 nm to about 250 nm and can comprise a magnetic core (e.g., a magnetite or iron core) with one or few nanolayers of silica layered thereon in order to form a binding support configured to efficiently bind nucleic acids (DNA and RNA) thereto. These magnetic particles are nonspecific capture elements and are target-independent based on the chemistry they are included within. Due to their extremely small size and homogeneous shape, the magnetic particles can be fully dispersed in any applicable solution, allowing more thorough nucleic acid binding, washing, and elution. The efficient purification of nucleic acids from a biological sample, free from interfering substances, coupled with high recovery, provide high-quality nucleic acids (e.g., DNA and RNA) for subsequent molecular analysis. First magnetic particles 108A can be provided as a suspension in a suitable liquid, and may be mixed by vortexing in a vortex mixer for a few minutes before aspiration to ensure substantially full suspension. First magnetic particles 108A can be as described in U.S. Pat. Nos. 9,617,534 and 10,385,331, for example. First magnetic particles 108A can be added in an amount of from about 25 μL to about 35 μL, for example, or in the amount of from about 8.3 μL to about 11.7 μL per each 1 mL of the sample portion 117. Nuclease-free deionized water 126 may be added in an amount of from about 1.13 mL to about 1.38 mL, for example, or in the amount of from about 0.38 mL to about 0.46 mL per each 1 mL of the sample portion 117.

The first binding buffer 135 can be of the same or similar composition as the lysis buffer 119. In particular, the first binding buffer 135 can include one or more chaotropic agents that functions as a protein denaturant and a nucleic add protector in the extraction of nucleic acids from the cells. For example, the first binding buffer 135 can comprise one or more chaotropic agents selected from the group of guanidinium hydrochloride (NH₂C(═NH)NH₂.HCl), guanidinium thiocyanate (H₂NC(NH)NH₂.HSCN), urea (carbimide or CH₄NO₂), and combinations thereof. Concentration of the one or more chaotropic agents may be from about 0M to 6M, or even between 2M and 6M, for example. The first binding buffer 135 may be added in the amount of about 0.8 mL to about 1.2 mL, or in the amount of from 0.27 mL to 0.40 ml per 1 mL of the sample portion 117, for example. First binding buffer 135 can also include a salt compound and possibly also a surfactant that can be the same as described above for the lysis buffer 119.

First magnetic particles 108A, first binding buffer 135, lysed sample 118, and nuclease-free deionized water 126 can be mixed, such as in a vortex mixer, for about 15 seconds and then incubated in a first incubation for a sufficient time to adequately bond the first nucleic acid portion 137 of lengths 500 bp (the large nucleic acid fragments) to the first magnetic particles 108A. The first incubation may be conducted without added heat, i.e., at room temperature (e.g., 20° C. to 25° C.) in some embodiments. First incubation of the first bindable mixture 138 may continue for about 8 to 12 minutes, or other suitable time to accomplish the substantially complete binding of the large nucleic acid fragments having lengths 500 bp to the first magnetic particles 108A. For example, the first bindable mixture 138 contained in the first vessel 130 may be agitated such as by being placed on a lab roller and rolled (designated as vibration 131) for about 10 minutes to accomplish the second incubation. In the case of use of a 96 well extraction plate, any suitable means for mixing/agitation the first bindable mixture 138 may be used.

Thus, according to the method 300, in block 308, the first bindable mixture 138 is incubated in a first incubation step to bind a first nucleic acid portion 137 having lengths greater than or equal to 500 bp to the first magnetic particles 108A and leave a first supernatant 139 as shown in FIG. 1E. First supernatant 139 includes the retained nucleic acid (e.g., cfDNA or RNA) with lengths <500 bp. Substantially all nucleic acid that has lengths 500 bp can be substantially removed in the first binding or negative selection step using the first magnetic particles 108A and first binding buffer 135 comprising a chaotropic agent, buffering agent, and a surfactant. The second nucleic acid portion 146 that is <500 bp is retained in the first supernatant 139.

Following the first incubation in 308, the first magnetic particles 108A are separated, in block 310, from the first supernatant 139. Separation can be by subjecting the magnetic particles 108A with bound first nucleic acid portion 137 having lengths greater than or equal to 500 bp to a suitable magnetic field. Magnetic field may be produced by any suitable magnetic separator device that includes a first magnet 140A that can be a moveable permanent magnet or optionally an electromagnet, having a magnetic field that can be selectively turned on and off. The magnetic field of the first magnet 140A is of sufficient strength to move the magnetic particles 108A, such as to one or more sides of the first vessel 130 as shown in FIG. 1E, so as to allow relatively clear access by the pipette 104 in order to aspirate the first supernatant 139 containing the second nucleic acid portion 146 having lengths less than 500 bp containing the mutations. The separation is completed as substantially all the aspirated first supernatant 139 from the first vessel 130 is transferred to a second vessel 142 as shown in FIG. 1F. Arrow 143 denotes dispensing of the first supernatant 139 into the second vessel 142 via the pipette 104 or other pipette.

Following separation in block 310, second magnetic particles 1086 are added to the dispensed first supernatant 139 along with a second binding buffer 144 to form a second bindable solution 145 as shown in block 312 and FIG. 1G. The second magnetic particles 1086 may be fresh (unbound) particles of the same type as the first magnetic particles 108A. The second magnetic particles 108B may be added to the first supernatant 139 in an amount of from about 5 μL to about 55 μL, or about 30 μL to about 55 μL, or from about 10 μL to about 18.3 per each 1 mL of the sample portion 117, for example. Other suitable amounts may be added.

Second binding buffer 144 can comprise an alcohol comprising isopropanol, ethanol, or a combination, in combination with a salt compound such as NaCl, a metal halide salt (e.g., potassium chloride (KCl)), a phosphate salt such as sodium phosphate, potassium phosphate such as monopotassium phosphate (KH₂PO₄) or dipotassium phosphate (K₂HFO₄), or a combination thereof. The salt concentration of second binding buffer 144 can be to be from about 1M to 6M, even from 2M to 5M in some embodiments. For the binding of small nucleic acids (<500 bp), the alcohol concentration of second binding buffer 144 can be made to be from about 15% to about 80% in some embodiments, or from about 40% to about 80%, even from about 50% to about 70% in other embodiments. The alcohol can function to remove the hydration shell of H₂O molecules around the phosphate. The salt compound can function to increase the ionic strength in order to substantially neutralize the negative charge of the nucleic acid chain. The total effect is that the small nucleic acid molecules (<500 bp) can come together due to neutralization of charge and removal of water that makes them relatively easier to bind to the silica surface of the second magnetic particles 1086.

Second binding buffer 144 is operative to assist in efficiently binding the second nucleic acid portion 146 (i.e., DNA or RNA fragments of lengths <500 bp containing the mutations) to the second magnetic particles 108B in a second binding step. The second binding buffer 144 may be added in an amount of from about 2.9 mL to about 3.6 mL, or from about 0.97 mL to about 1.20 mL per 1 mL of the sample portion 117. In some embodiments, the second binding buffer 144 can comprise isopropanol and a salt. For example, in one embodiment, the second binding buffer 144 can be made up of about 2 mL of 15% to 45% isopropanol and about 1 mL of 5M NaCl.

Upon addition of the second magnetic particles 1086 and the second binding buffer 144 to the first supernatant 138, the second bindable mixture 145 is incubated, in block 314, in a second incubation phase to bind a second nucleic acid portion 146 having lengths <500 bp to the second magnetic particles 108B and leave a second supernatant 148. The second incubation phase can be conducted for a time sufficient to substantially fully bind the second nucleic acid portion 146 having length <500 bp to the second magnetic particles 1086. For example, in the second incubation phase, the second bindable mixture 145 of second magnetic particles 108B, second binding buffer 144, and first supernatant 139 can be capped or covered, mixed in the second vessel 142, such as on a vortex mixer, for about 15 seconds, and then incubated for about 8 minutes to 12 minutes at room temperature (from 20° C. and 25° C.), for example. Second incubation may be undertaken while being gently agitated, such as by rolling or by other suitable agitation device, and thus may be mixed, rolled, or otherwise agitated as indicated by vibration 131 during the second incubation.

Following the completion of the second incubation in block 314, the second magnetic particles 108B with the second nucleic acid portion 146 bound thereto are separated from the second supernatant 148 as shown in FIG. 1H and block 316. The separation can involve using a suitable second magnet 140B to attract the second magnetic particles 1086 to one or more sides of the second vessel 142 and then aspirating the second supernatant 148 from the second vessel 142 with pipette 1054 or other pipette as shown in FIG. 1I. Second supernatant may be discarded. The second magnet 140A may be the same or different magnet than first magnet 140A. For example, in some embodiments, magnet 140A may be moveable from the location of the first vessel 130 to the location of the second vessel 142, for example.

According to the sample preparation method 300, the second magnetic particles 108B with the second nucleic acid portion 146 bound thereto are then washed in a washing step as shown in FIG. 1I-1K and block 318. Washing step can take place at a wash station 147 that is configured to carry out first and second wash phases of the second magnetic particles 108B with bound second nucleic acid portion 146 in two wash phases. The wash station 147 may contain, in close proximity, the first and second wash buffers 149A, 149B, a magnet 140C, and some member for providing agitation, and a disposal reservoir (not shown), for example. Member for providing agitation may be a vibrating pipette, or an ultrasonic vibrator for agitating the wash buffers and second magnetic particles 1086. The pipette 104 or another dedicated pipette or pipettes can be located at the wash station during washing phases. Magnet 140C may be a dedicated magnet like magnet 140A, or the magnet 140A may be a moveable magnet that can be moved to the location of the wash station 147 by any suitable mechanism.

After aspiration of the supernatant 148 via pipette 104 or other pipette, the first wash phase can include immersing the second magnetic particles 1086 with bound second nucleic acid portion 146 in a first wash buffer 149A (FIG. 1J). For example, the first wash phase may include dispensing the first wash buffer 149A until the second magnetic particles 108B are immersed, agitating via vortexing (e.g., via vibration 131), by a suitable agitation member and then aspiration of the remaining first wash buffer/supernatant after washing. Aspiration can occur after moving the second magnetic particles aside via the magnetic field produced by the third magnet 140C for a suitable amount of time to produce a clear buffer/supernatant. The first wash buffer/supernatant can be discarded. The first wash buffer 149A may be provided in an amount of from 0.8 mL to 1.2 mL, or in an amount of about 0.27 mL and 0.40 mL per 1 mL on sample portion 117. First wash buffer 149A may be a solution comprising a chaotropic agent, a salt compound, and an alcohol. For example, the first wash buffer 149A may be made up of 3M guanidinium-based compound, 100 mM sodium acetate, and 30% ethanol.

This can be followed by a second wash phase involving dispensing and immersing the second magnetic particles 108B in a second wash buffer 149B (FIG. 1K). First and second wash buffers 149A, 149B may be different. The second wash buffer 149B can be a solution comprising a salt compound and an alcohol. For example, the second wash solution can comprise a composition made up of 10 mM sodium acetate and 80% ethanol. The second wash buffer 149B may be provided in an amount of from 0.8 mL to 1.2 mL, or in an amount of about 0.27 mL and 0.40 mL per 1 mL on sample portion 117. For example, the second wash phase may include dispensing the second wash buffer 149B, agitation 131 with a member, and then aspiration of the remaining second wash buffer/supernatant. The second wash buffer/supernatant can be discarded. Aspiration can occur after moving the second magnetic particles 1086 aside via the magnetic field produced by the third magnet 140C. Aspiration can occur when the buffer/supernatant is clear. Additional wash phases could be implemented.

Following the wash phases in block 318, an elution buffer 150 is added to the second magnetic particles 108B with bound second nucleic acid portion 146 in the second vessel 142 as shown in FIGS. 1L and block 320. The elution buffer 150 can be a composition comprising hydroxymethyl aminomethane hydrochloride (Tris-HCl), or optionally a composition comprising Tris-HCI and ethylenediaminetetraacetic acid (EDTA). For example, the Tris-HCL can have a pH from 7 to10 and molarity of from 1mM to 100 mM, or even of 0.5 mM to 20 mM. EDTA can comprise a molarity of 0 mM to 10 mM, or even 0 mM to 5 mM in some embodiments. In one example, the elution buffer 150 can comprise 10 mM Tris-HCL and 0.1 mM EDTA.

The elution buffer 150 can be aspirated and dispensed by the pipette 104 or another pipette at a location of an elution stage 151 (FIG. 1M), and may be provided in the second vessel 142 in an amount of between about 90 μL and 110 μL, or between 30 μL and 37 μL for each mL of the sample portion 117, for example. The mixture of elution buffer 150 and second magnetic particles 1086 with bound second nucleic acid portion 146 are then incubated in a third incubation in block 320 to release the second nucleic acid portion 146 into, and form, a third supernatant (the final eluate 152) as shown in FIG. 1M. For example, the third incubation may be conducted for from about 8 minutes to about 12 minutes at from about 25° C. to about 80° C., or even from 25° C. to about 45° C., in some embodiments. Third incubation can involve supplying heat 143H from a heating element 143 at the elution state 151. Heating element 143 may be the same or similar as heating element 134. In the case of the sample preparation method taking place on a 96 well extraction plate, the heating element 143 can be a heater block adapted to heat the desired wells simultaneously. The third supernatant is the final eluate 152 produced by the sample preparation method 300 and has the second nucleic acid portion 146 with lengths of less than 500 bp contained therein.

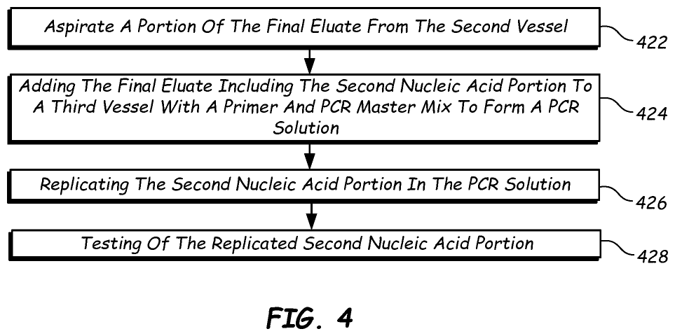

Once sample processing on the first and second vessels 130, 142 is completed for a particular sample portion 117 of a biological sample 112, the third supernatant (final eluate 152) can be extracted and further processed. For example, the second magnetic particles 108B can be pulled aside by fourth magnet 140D at the elution stage 151 so that a selected amount of the final eluate 152 can be aspirated by pipette 104 or another pipette as shown in FIG. 1N and in block 422 of FIG. 4. Fourth magnet 140D can be the same as magnet 140A or may be a moveable magnet.

Now as should be understood, the sample preparation system 200 is adapted to prepare a biological sample 112 for further PCR processing. The sample preparation system 200 comprises a kit 275, a collection of consumable solutions or suspensions, comprising a lysis agent 119, a first binding buffer 135 comprising one or more chaotropic agents, a salt compound, and a surfactant, a second binding buffer 144 comprising isopropanol, ethanol, sodium chloride, potassium chloride, sodium phosphate, potassium phosphate, or combinations thereof, magnetic particles 108A, 1086 operable as binding supports, a first wash buffer 149A comprising a chaotropic agent, a salt compound, and an alcohol, a second wash buffer 149B comprising a salt compound and an alcohol, and an elution buffer 150 comprising TRIS-HCL.

The sample preparation system 200 further comprises the first vessel 130 positioned to receive the sample portion 117 of the serum or plasma portion 114 of the biological sample 112 containing nucleic acids 106 and the lysis agent 119, and a heater element 134 operable to heat the sample portion 117, lysis agent 119 and possibly a protein removal agent 120, and form the lysed sample 118.

Sample preparation system 200 further comprises the pipette 104 coupled to the aspiration and dispensing apparatus 220 and is configured and operable to aspirate and dispense the first magnetic particles 108A (contained in a liquid suspension) and the first binding buffer 135 into the lysed sample 118 and form a first bindable mixture 138, which upon a first incubation binds a first nucleic acid portion 137 having lengths greater than or equal to 500 bp to the first magnetic particles 108A and leaves a first supernatant 139.

The sample preparation system 200 further comprises the magnet 140 operable to separate the first magnetic particles 108A with bound first nucleic acid portion 137 from the first supernatant 139. A second vessel 142 of the sample preparation system 200 receives the first supernatant 139, the second magnetic particles 108B, and the second binding buffer 144 (via aspiration and dispense by pipette 104 or other pipette), which upon a second incubation binds a second nucleic acid portion 146 having lengths less than 500 bp to the second magnetic particles 108B and leaves a second supernatant 148.

Sample preparation system 200 can further comprise a second magnet 140B configured to separate the second magnetic particles 108B with the second nucleic acid portion 146 bound thereto from the second supernatant 148.

The sample preparation system 200 further comprises a wash station 147 configured to carry out first and second wash phases of the second magnetic particles 1086 with bound second nucleic acid portion 146, after separation from the second supernatant 148. The first wash phase can comprise immersing the second magnetic particles 1086 with a first wash buffer 149A and the second wash phase comprises immersing the second magnetic particles 108B with a second wash buffer 149B. Immersion can be via dispense of the and first wash buffer 149A and the second wash buffer 149B by pipette 104 or another pipette or pipettes.

Further, the sample preparation system 200 can comprise an elution stage 151 or location wherein the elution buffer 150 is added to the second magnetic particles 1086 after the first and second wash phases and incubated in a third incubation to release the second nucleic acid portion 146 and form final eluate 152.

In some embodiments, this final eluate 152 can be added (dispensed) in a desired volume into one or more third vessels such as test vessel 154 (e.g., PCR test vessel) in block 424. The final eluate 152 contains both small nucleic acids including DNA and RNA having lengths <500 bp. DNA can be analyzed by itself or DNA and RNA can be analyzed simultaneously by implementing an intermediate RT-PCR step that converts RNA into copyDNA and from there everything is DNA for further amplification and analysis. In the case of PCR processing, a PCR master mix 156 and primer and/or probe 158, and possibly a reagent and/or water, may also be added in block 424 to produce a PCR solution 159. The next stages of the processing method can involve replication (amplification) and analysis. Replication (amplification) of the DNA templates extracted in the sample preparation method 300 (i.e., the second nucleic acid portion 146 containing DNA with lengths less than 500 bp) involves making millions of copies of the nucleic acid templates 146. Thereafter, analysis (testing) of the replicated PCR solution involves detection (e.g., fluorescence detection) with a detection system 170, for example. Depending on the particular type of processing of the nucleic acids (e.g., DNA only or DNA plus converted RNA) that will take place, other steps such as an index-ligation step or a reverse transcriptase step can be conducted before PCR.

As shown in FIGS. 1N and 1O, a portion of the third supernatant (final eluate 152) can be transferred from the second vessel 142, such as by aspiration with the pipette 104 or other pipette and coupled aspiration and dispense apparatus 220, to a test vessel 154. In the case where the replication is one of many parallel PCR processes taking place on a PCR test plate, one, or more than one, test plate well of a PCR test plate may be populated with a portion of the final eluate 152. For example, different assays may be conducted using the final eluate 152. The act of transfer is designated by arrow 155, wherein robot 205 and coupled pipette 104 or other pipette aspirates and then dispenses the portion of the third supernatant (final eluate 152) into the PCR test vessel 154. PCR test vessel 154 could be any suitable vessel having transparent or translucent walls and may be a well of a PCR test plate including multiple wells (e.g., 96 wells).

Along with the portion of the final eluate 152, a PCR master mix 156 may be added along with a suitable primer and/or probe 158. Primer or probe (or primer probe mix) 158 for those protocols desiring primer and probe may be added to the test vessel 154. Likewise, enzyme for those protocols desiring enzyme may be added to the test vessel 154. Thus, a PCR solution 159 for processing is provided in the test vessel 154. A desired number of heating and cooling cycles may be applied to the PCR solution 159 in the test vessel 154 by any suitable heating and cooling apparatus. For example, heating apparatus 160 may produce heat 161 that heats the PCR solution 159 to an annealing temperature of above about 80° C. Thereafter, the PCR solution 159 may be cooled by extracting heat 162 by operation of a cooling apparatus 164 to below about 65° C. Other suitable temperatures may be used depending on the primers or probes used. Any suitable construction of the heating apparatus 160 and cooling apparatus 164 can be used. The heating and cooling cycles operate, in block 426, to replicate the second nucleic acid portion 146 (small DNA templates having lengths <500 bp) contained in the PCR solution 159.

Upon completion of a predesigned number of heating and cooling cycles, the PCR method includes analysis of the amplified PCR solution 165. In the case where one or more fluorescent dyes are tagged to the nucleic acid templates, a detection apparatus 170 can be used for the analysis. The detection apparatus 170 can include a light source 172 for producing excitation light at one or more wavelengths and a light detector 174 that can detect light emissions excited by the light excitation. Any suitable configuration of the detection apparatus 170 may be used, such as known fluorescence detection apparatus. Thus, the detection apparatus 170 operates to test the second nucleic acid portion 146 in the amplified PCR solution 165 in block 428.

Table 1 below illustrates example results of the relative concentrations of 500 bp to1000 bp DNA as compared to concentrations of 100 bp to 300 bp DNA that are present after the PCR processing. For the experiment, spike-in DNA (170 bp to180 bp PCR product was added to the blood sample and we used a competitive, commercially available cfDNA extraction kit as a standard (competitive). Table 1 illustrates that DNA obtained from the first binding of the present 2-step method 300 is mainly the large molecules (>500 bp), wherein the concentration of DNA 500 bp to 1000 bp is dropped to 80.5 ng/m L. This advantageously amounts to 60% less DNA from 500 bp to 1000 bp than the competitive method.

The DNA obtained from the second binding of the 2-step method 300 is mainly small molecules (<500 bp), but without much further downward change in the concentration of large length DNA (500 bp to 1000 bp). For example, in the second binding, 226 ng/mL of the desired DNA 100 bp to 300 bp is extracted, which advantageously is about 6% more than the competitive method. However, more significant is the much smaller amount of large DNA present such that a concentration ratio of the small DNA concentration divided by large DNA concentration (i.e., the concentration of DNA 100 bp to 300 bp divided by the concentration of DNA 500 bp to1000 bp). In the depicted example, the concentration ratio is less than 1.0 for the competitive example, but greater than 2.0, or even greater than 2.5 in the present method 300. Therefore, the relative amount of large DNA 500 bp is much less in the present method 300 (78.8 ng/mL versus 224 ng/mL). This dramatically lowers the background noise caused by the presence of the 500 bp to 1000 bp DNA and improves ability to properly analyze any mutations in the 100 bp to 300 bp range.

TABLE 1
Examples

| | DNA Concentration | DNA Concentration | |
|---|---|---|---|
| | (100 bp to 300 bp) | (500 bp to 1000 bp) | |
| Sample | (ng/mL) | (ng/mL) | Ratio |
| 2-Step Method | 16.4 | 80.5 | na |
| (First Binding) | | | |
| 2-Step Method | 226 | 78.8 | 2.87 |
| (Second Binding) | | | |
| Competitive Method | 212 | 224 | 0.95 |

### Example Manual Two-Part Sample Preparation Method

Materials that can be used for the manual sample preparation method are:

15 mL centrifuge tubes

1.5 mL micro-centrifuge tubes

Thermostat (e.g., Lauda RM6 temperature thermostat or equivalent)

Thermomixer (e.g. Eppendorf Thermomixer, or equivalent)

Universal Centrifuge (e.g., Hettich Universal Centrifuge or equivalent)

Vortex mixer (e.g. IKA Vortex Mixer or equivalent)

Mini Labroller (Labnet Inernational or equivalent)

Magnetic Separator (Miltenyl Biotec Sepaarator or equivalent)

Microcentrifuge (e.g. Eppendorf miniSpin or equivalent)

Magnetic stand (e.g. Promega magnetic rack or equivalent)

A kit 275 adapted to preparation of a biological sample for PCR processing.

### Kit

In particular the kit 275 (FIG. 2) adapted for use with the method can comprise:

a lysis agent 119 configured to lyse a sample portion 117 of the biological sample 114;

a first binding buffer 135 comprising one or more chaotropic agents, a salt compound, and a surfactant.

a second binding buffer 144 comprising an alcohol of isopropanol, ethanol, or a combination thereof, and a salt compound comprising sodium chloride, potassium chloride, sodium phosphate, potassium phosphate, or a combination thereof;

magnetic particles 108A, 108B operable as binding supports;

a first wash buffer 149A comprising a chaotropic agent, a salt compound, and an alcohol;

a second wash buffer 149B comprising a salt compound and an alcohol; and an elution buffer 150 comprising TRIS-HCL.

### Manual Two-Part Procedure

The following method may be used to prepare the final eluate 152 having nucleic acids of lengths <500 bp.

1) Pre-warm the temperature of each of Thermostat and the Thermomixer to about 37° C.

2) Place biological sample 112 (e.g., EDTA blood sample) contained in the blood collection tube 110 into the Universal Centrifuge. Centrifuge at room temperature for 10 minutes at 2,000×g to separate the plasma portion 114 from the red blood cell portion 116. Note: Other blood collection tubes 110 (e.g. Streck tubes with K₃EDTA) and established centrifugation conditions therefor can be used instead.

3) Carefully transfer 3 mL of the serum or plasma portion 114 (e.g., plasma) from blood collection tube 110 to an intermediate sample tube (e.g., a 15 mL centrifuge tube).

4) Centrifuge the serum or plasma portion 114 at room temperature for 10 minutes at 5,000×g.

5) Carefully transfer 3 mL of the serum or plasma portion 114 (e.g., plasma) from intermediate sample tube to a first vessel 130 (e.g., a new 15 mL centrifuge tube).

6) Add 100 μL of the protein removal agent 120 (e.g., Proteinase K) to the first vessel 130, and then cap the first vessel 130 and vortex with the Vortex Mixer.

7) Add 3.75 mL of the lysis buffer 119. Cap the first vessel 130 and vortex with the Vortex Mixer.

8) Incubate the solution of sample portion 117, lysis buffer 119, and protein removal agent 120 in the first vessel 130 on the Thermostat at about 37° C. for about 10 minutes.

9) After the first incubation is complete, remove the first vessel 130 containing the lysed sample 118 from the Thermostat.

10) Vortex suspension including the first magnetic particles 108A in the Vortex Mixer for 2 minutes before use.

11) Add 30 μL of the first magnetic particles 108A from the vortexed suspension into the first vessel 130, 1 mL of the first binding buffer 135, and further add 1.25 mL nuclease-free water to the lysed sample 118. Cap the first vessel 130 and vortex with the Vortex Mixer.

12) Install the first vessel 130 (e.g., 15 mL sample tube) on Mini Labroller and roil for about 10 minutes at room temperature to accomplish the second incubation.

13) Centrifuge tubes for 5 seconds at 2,000×g to minimize carry over when opening cap.

14) Transfer the first vessel 130 to the Magnetic Separator to separate first magnetic particles 108A and first supernatant 139.

15) While still on the Magnet Separator, aspirate the first supernatant 139 with a pipette to a second vessel 142 (e.g., a new 15 mL tube).

16) Add 50 μL of second magnetic particles 108B from the vortexed magnetic particle suspension into the second vessel 142 and add second binding buffer 144 (e.g., 1 mL 5M NaCl and 2 mL isopropanol) to the first supernatant 139 in the second vessel 142. Cap the second vessel 142 and vortex on Vortex Mixer.

17) Install the second vessel 142 on Mini Labroller and roll for 10 minutes at room temperature to accomplish a second incubation.

18) Centrifuge the second vessel 142 for 5 seconds at 2,000×g to avoid carry over when opening caps.

19) Transfer the second vessel 142 to Magnetic Separator to separate the second magnetic particles 1086 from the second supernatant 148.

20) While still on the Magnetic Separator, aspirate second supernatant 148 with a pipette and discard.

21) Add 1 mL of the first wash buffer 149A and suspend the second magnetic particles 1086 by vortexing. Transfer the second magnetic particles 1086 carefully to a new microcentrifuge tube (e.g., 1.5 mL microcentrifuge tube).

22) Transfer the microcentrifuge tube to a Magnetic Stand and magnetize until wash buffer/supernatant is clear.

23) While still on the Magnetic Stand, remove wash buffer/supernatant with a pipette to the 15 mL sample tube and vortex to collect the rest of the second magnetic particles 1086. Transfer the rest of the suspension carefully to the microcentrifuge tube on the Magnetic Stand.

24) Magnetize until supernatant is clear. Remove wash buffer/supernatant with a pipette and discard.

25) Add 1 mL of a second wash buffer 149B and suspend the second magnetic particles 108B by vortexing.

26) Transfer the microcentrifuge tube back to Magnetic Stand.

27) Remove wash buffer/supernatant with a pipette and discard.

28) Add 100 μL of the elution buffer 150 and vortex to suspend the second magnetic particles 108B.

29) Incubate the supernatant in the thermomixer at about 37° C. with agitation at about 1100 rpm for 10 minutes to unbind the second DNA portion having lengths <500 bp from the second magnetic particles 108B.

30) Centrifuge eluate 152 briefly (about 5 seconds) at 15000×g to remove any liquid from the cap.

31) Transfer to Magnet Stand to separate second magnetic particles 108B. Magnetize until third supernatant (final eluate 152) is visually clear.

32) Transfer final eluate 152 containing total nucleic acids into a PCR test vessel 154 with sample IDs. Optionally, store eluate 152 at −80° C. until use.

### DEFINITIONS

Lysis Buffer—A chemical compound that is a buffer solution used for the purpose of breaking open cells of a biological sample for use in molecular biology testing that analyzes the labile macromolecules of the cells.

Lysate or Lysed Sample—A preparation containing the products of lysis of cells.

Binding Buffer—A solution that is added to a quantity of mixture containing cell nucleic acids and binding supports to produce conditions that enable the nucleic acids to bind to a surface of the binding support, such as a silica-coated magnetic particle.

Elution Buffer—Is a solution used to release a desired nucleic acid from the binding support (e.g., silica-coated magnetic particles) without appreciably changing the function or activity of the desired protein.

Eluate—a substance (e.g., a target nucleic acid) separated out by, or the product of, elution or elutriation.

Surfactant—Can be a detergent or emulsifier that does not substantially interfere with the nucleic acid binding to the binding support (e.g., silica-coated magnetic particles), but it helps disperse the molecules. Further, the surfactant can help reduce nonspecific binding to the vessel/well by saturating those possible sites.

Pre-processed control—A process control that has been processed along with the biological sample portion and then are transferred for further molecular processing along with the final eluate.

Post-processed control—A process control that has been processed by the manufacturer and that gets directly loaded into a PCR test well (e.g., of a PCR test plate) along with final eluate, PCR master mix, and primer or probe.

Internal control—A process control that is added to a patient samples portion that indicate the sample preparation process has proceeded without any reaction issues that interfere with the end result.

Proteinase K—Proteinase K is a broad-spectrum serine protease. Proteinase K is commonly used in molecular biology to digest protein and remove contamination from preparations of nucleic acid. Addition of Proteinase K to nucleic acid preparations rapidly inactivates nucleases that might otherwise degrade the DNA or RNA during purification.

Master mix—Master mix is premixed, ready-to-use solution containing polymerase components and other components (e.g., Taq DNA polymerase, dNTPs, MgCl₂ and reaction buffers) at optimal concentrations for efficient amplification of nucleic acid templates (e.g., DNA and RNA templates).

The foregoing description discloses only example embodiments of the disclosure. Modifications of the above-disclosed methods, kits, and apparatus and which fall within the scope of the disclosure will be readily apparent to those of ordinary skill in the art. Accordingly, while the present disclosure has been disclosed in connection with example embodiments contained herein, it should be understood that other alternative embodiments may fall within the scope of the disclosure, as defined by the following claims.

What is claimed is:

1. A method of extracting nucleic acid from a biological sample, comprising:
providing a sample portion of the biological sample containing the nucleic acid to a first vessel;
causing lysis of the sample portion to form a lysed sample;
adding first magnetic particles to the lysed sample along with a first binding buffer to form a first bindable mixture;
incubating the first bindable mixture in a first incubation to bind a first nucleic acid portion having lengths greater than or equal to 500 bp to the first magnetic particles and leave a first supernatant;
separating the first magnetic particles from the first supernatant;
adding second magnetic particles to the first supernatant along with a second binding buffer to form a second bindable mixture;
incubating the second bindable mixture in a second incubation to bind a second nucleic acid portion having lengths less than 500 bp to the second magnetic particles and leave a second supernatant;
separating the second magnetic particles with the second nucleic acid portion bound thereto from the second supernatant;
washing the second magnetic particles with second nucleic acid portion bound thereto; and
adding an elution buffer to the second magnetic particles after the washing and incubating in a third incubation to release the second nucleic acid portion and form a third supernatant.

2. The method of claim 1, wherein the lysis of the sample portion is provided by adding a lysis buffer, serine protease, and heating for a lysis period to form the lysed sample.

3. The method of claim 2, wherein the lysis buffer comprises one or more chaotropic agents comprising:
urea (CH₄N₂O),
a guanidinium-based compound,
or a combination thereof.

4. The method of claim 2, wherein the lysis buffer comprises a guanidinium-based compound and a salt compound.

5. The method of claim 1, wherein the first magnetic particles and the second magnetic particles comprise a magnetite core with a silica coating.

6. The method of claim 1, wherein the first magnetic particles are added in an amount of from 8.3 μL to 11.7 μL per each 1 mL of the sample portion.

7. The method of claim 1, wherein the first binding buffer comprises one or more chaotropic agents.

8. The method of claim 7, wherein the first binding buffer further comprises a salt compound and a surfactant.

9. The method of claim 8, wherein the first binding buffer is added in an amount of from 0.27 mL and 0.40 ml per 1 mL of the sample portion.

10. The method of claim 8, wherein the incubating of the first bindable mixture in the first incubation is carried out at a first incubation temperature of from 20° C. to 25° C. for a first incubation period from 8 minutes to 12 minutes.

11. The method of claim 1, wherein the separating of the first magnetic particles from the first supernatant, comprises:
subjecting the first magnetic particles to a magnetic field to move the first magnetic particles aside in the first vessel; and
aspirating and transferring the first supernatant containing the second nucleic acid portion having lengths less than 500 bp to a second vessel, while leaving behind the first magnetic particles.

12. The method of claim 1, wherein the second binding buffer comprises:
an alcohol comprising isopropanol, ethanol, or a combination thereof; and
a salt compound comprising sodium chloride, potassium chloride, sodium phosphate, potassium phosphate, or a combination thereof.

13. The method of claim 12, wherein the second binding buffer comprises a combination of isopropanol and sodium chloride.

14. The method of claim 1, wherein the separating of the second magnetic particles from the second supernatant comprises aspirating the second supernatant while leaving behind the second magnetic particles with bound second nucleic acid portion.

15. The method of claim 1, wherein the washing of the second magnetic particles with the second nucleic acid portion bound thereto comprises a first wash phase of immersing the second magnetic particles in a first wash buffer comprising a chaotropic agent, a salt compound, and an alcohol.

16. The method of claim 15, wherein the washing of the second magnetic particles further comprises a second wash phase following the first wash phase comprising immersing the second magnetic particles in a second wash buffer comprising a salt compound and an alcohol.

17. The method of claim 1, wherein the elution buffer comprises Tris-HCL.

18. The method of claim 17, wherein the Tris-HCL has pH from 7 to10 and molarity of 0.5 mM to 20 mM.

19. The method of claim 17, further comprising EDTA having a molarity of 0 mM to 5 mM.

20. A kit adapted to preparation of a biological sample for further diagnostic processing, the kit comprising:
a lysis agent configured to lyse the biological sample;
a first binding buffer comprising one or more chaotropic agents, a salt compound, and a surfactant;
a second binding buffer comprising:
an alcohol comprising isopropanol, ethanol, or a combination thereof, and
a salt compound comprising sodium chloride, potassium chloride, sodium phosphate, potassium phosphate, or a combination thereof;
magnetic particles operable as binding supports;
a first wash buffer comprising a chaotropic agent, a salt compound, and an alcohol;
a second wash buffer comprising a salt compound and an alcohol; and
an elution buffer comprising Tris-HCL.

21. A sample preparation system adapted to prepare a biological sample for molecular processing, comprising:
a kit comprising a lysis agent, a first binding buffer comprising one or more chaotropic agents, a salt compound, and a surfactant, a second binding buffer comprising: an alcohol comprising isopropanol, ethanol, or a combination thereof, and a salt compound comprising sodium chloride, potassium chloride, sodium phosphate, potassium phosphate, or a combination thereof, magnetic particles operable as binding supports, a first wash buffer comprising a chaotropic agent, a salt compound, and an alcohol, a second wash buffer comprising a salt compound and an alcohol, and an elution buffer comprising TRIS-HCL;
a first vessel positioned to receive a sample portion of the biological sample containing nucleic acid and the lysis agent;
a heater element operable to heat the sample portion and the lysis agent and form a lysed sample;
a pipette coupled to an aspiration and dispensing apparatus configured and operable to aspirate and dispense first magnetic particles and the first binding buffer into the lysed sample and form a first bindable mixture, which upon a first incubation binds a first nucleic acid portion having lengths greater than or equal to 500 bp to the first magnetic particles and leaves a first supernatant;
a first magnet operable to separate the first magnetic particles with bound first nucleic acid portion from the first supernatant;
a second vessel receiving the first supernatant, second magnetic particles, and the second binding buffer, which upon a second incubation binds a second nucleic acid portion having lengths less than 500 bp to the second magnetic particles and leaves a second supernatant;
a second magnet separating the second magnetic particles with the second nucleic acid portion bound thereto from the second supernatant;
a wash station configured to carry out first and second wash phases of the second magnetic particles with bound second nucleic acid portion, after separation from the second supernatant, wherein the first wash phase comprises immersing the second magnetic particles with a first wash buffer and the second wash phase comprises immersing the second magnetic particles with a second wash buffer; and
an elution stage wherein the elution buffer is added to the second magnetic particles after the first and second wash phases and incubated in a third incubation to release the second nucleic acid portion and form a final eluate.
